(19) United States
(12) Patent Application Publication
(10) Pub. No.: US 2007/0197868 A1
(43) Pub. Date: Aug. 23, 2007

(54) Self-propelled colonoscope

(76) Inventor: Masazumi Takada, Matsudo-City (JP)

Correspondence Address: YOUNG & THOMPSON, 745 SOUTH 23RD STREET, 2ND FLOOR, ARLINGTON, VA, 22202, US

(21) Appl. No.: 11/705,735

(22) Filed: Feb. 14, 2007

(30) Foreign Application Priority Data
Feb. 17, 2006 (JP) 2006-040257

(51) Int. Cl. A61B 1/00

(52) U.S. Cl. 600/114; 600/101; 600/139

(57) ABSTRACT

A self-propelled colonoscope includes a tubular flexible insertion portion inserted into the colon, an endless belt arranged on a circulating path around the outer and inner walls of the flexible insertion portion, a driving mechanism to drive the endless belt, and a guide pipe extending along the circulating path and guiding the endless belt inside the flexible insertion portion. At the distal end of the flexible insertion portion, the guide pipe passes through a guide hole penetrating the flexible insertion portion to the outside, and the flange section formed at the distal end of the guide pipe adheres to the outer wall of the flexible insertion portion. Accordingly, the body of the colonoscope (especially the insertion tube) can be kept from being contaminated when the endless belts are driven or the body of the colonoscope is cleaned.

### FIELD OF THE INVENTION

The present invention relates to a self-propelled endoscope inserted into the colon by endless driving belts, arranged on the inner and outer walls of a flexible section of the insertion tube in a loop fashion. Particularly, it relates to a self-propelled colonoscope with improved liquid-tight ability via a hole in the flexible section, through which the endless belt penetrates the insertion tube from inside to outside.

### BACKGROUND OF THE INVENTION

Colonoscopic examination is currently carried out by inserting a colonoscope into the colon by hand. Since the colonoscope has to be inserted around the curves of the colon, the examination may cause the patient pain by excessively extending or bending the colon. As a colonoscope which does not cause a patient pain, self-propelled colonoscopes have been proposed.

The inventor proposed a self-propelled colonoscope equipped with endless belts arranged on the inner and outer walls of a flexible section of the insertion tube in a loop fashion and inserted into the colon by driving the endless belts (Japanese Patent No. 3514252). The endless belts run in the opposite direction to the insertion direction of the colonoscope, contacting the inner wall of the colon outside the flexible section and propelling the colonoscope into the colon. Inside the flexible section, the endless belts run through guide pipes, mounted on the inner wall of the flexible section extending in the length direction of the flexible section. At the distal end of the flexible section, the endless belts pass through guide holes, penetrating from the inner to the outer wall of the flexible section, and exits the guide pipes. By driving the endless belts with a driving mechanism, the endless belts propel the colonoscope into the colon by friction between the endless belts and the inner wall of the colon outside the flexible section. Inside the flexible section, the endless belts run through the guide pipes smoothly, so the colonoscope can be propelled into the colon without excessive extension and bending of the colon. Therefore, the colonoscope can be smoothly inserted into the colon while keeping the colon in relatively the same position and shape, causing the patient hardly any pain.

Since the endless belt can be detached at the intermediate portion, when the colonoscope is cleaned, the endless belt is detached and removed from the colonoscope. The endless belt and the body of the colonoscope are cleaned separately.

The flexible section of the insertion tube of the colonoscope is made of a flexible tube having a four-layer structure; a coating layer, a resin layer, a mesh layer and an inner and outer flex layer. When the flexible section with this structure has a guide hole penetrating from the inner to the outer wall, since the mesh layer and the flex layer are not solid but porous, liquid may infiltrate the layers from the sidewall of the guide hole. So, when the endless belts are driven, bodily fluid and matter adhered to the endless belts may infiltrate the layers from the sidewall through the guide hole and affect the inner components, such as wires, installed in the flexible section. Furthermore, the same problems may occur during the cleaning process after removing the endless belt.

In addition, when the mesh and flex layers are exposed, the inner components may suffer damages.

### SUMMARY OF THE INVENTION

The object of the invention is therefore to provide a self-propelled colonoscope with improved liquid-tight ability via a guide hole in the flexible section, through which the endless belt penetrates from inside to outside of the flexible section, in order to keep the body of the colonoscope (especially the insertion tube) from being contaminated when the endless belts are driven or the body of the colonoscope is cleaned.

The self-propelled colonoscope according to the present invention comprises a tubular flexible insertion portion inserted into the colon; an endless belt arranged on a circulating path around the outer and inner walls of the flexible insertion portion; a driving mechanism to drive the endless belt; and a guide pipe extending along the circulating path and guiding the endless belt inside the flexible insertion portion, where the end of the guide pipe penetrates through the tube wall of the flexible insertion portion to the outside at the distal end of the flexible insertion portion.

In the self-propelled colonoscope, the flexible insertion tube has a portion through which the endless belt passes from inside to outside at the distal end (through-hole). It is therefore necessary to make the sidewall of the through-hole liquid-tight because when the flexible insertion portion has a four-layer structure (coating layer, resin layer, mesh layer and flex layer) and a through-hole is made, liquid may infiltrate the mesh and flex layers from the sidewall of the through-hole since such layers are not solid but porous. Consequently, the self-propelled colonoscope is designed so that the guide pipe penetrates the through-hole and passes the outer wall of the flexible insertion portion. This prevents the mesh and flex layers from being exposed to the sidewall of the through-hole so that they can be kept from infiltration by bodily fluid adhered to the endless belt from the sidewall when it is driven. Similarly, when the body of the colonoscope is cleaned, the layers are not infiltrated with cleaning liquid from the sidewall, and the inner components, such as wires, installed in the flexible insertion portion, are not damaged.

In the present invention, the end of the guide pipe is formed into a flange shape, the end of which is adhered to the outer wall of the flexible insertion portion.

In this way, it becomes easy to attach the end of the guide pipe to the outer wall. And, since the flange increases the contact area between the end of the guide pipe and the outer wall of the flexible insertion portion, the seal between them is improved.

The self-propelled endoscope comprises: a tubular flexible insertion portion inserted into an object; an endless belt arranged on a circulating path around the outer and inner walls of the flexible insertion portion; a driving mechanism to drive the endless belt; and a guide pipe extending along the circulating path and guiding the endless belt inside the flexible insertion portion, where the end of the guide pipe penetrates through the tube wall of the flexible insertion portion to the outside at the distal end of the flexible insertion portion.

The self-propelled endoscope can be used in various fields such as disaster relief and industry by changing the thickness and length of the flexible insertion portion.

### EFFECT OF THE INVENTION

As described above, according to the present invention, the sidewall of the through-hole formed in the flexible insertion portion is liquid-tight so the layers constituting the flexible insertion portion are not infiltrated with bodily fluid adhered to the endless belt from the sidewall of the through-hole when it is driven. The layers are not infiltrated with cleaning liquid when the body of the colonoscope is cleaned.

### BRIEF DESCRIPTION OF THE DRAWINGS

FIG. 1 is a perspective drawing showing the self-propelled colonoscope (example).

FIG. 2 is a sectional drawing showing the distal section of the insertion portion.

FIG. 3 shows the structure of the flexible tube of the flexible section of the insertion portion.

FIG. 4 is a sectional drawing showing the flexible section of the insertion portion.

FIG. 5 is a side drawing showing a portion near the distal end of the flexible section.

FIG. 6 is an enlarged side sectional drawing showing a portion near the distal end of the flexible section.

FIG. 7(A) is a perspective drawing schematically showing the structure of the endless belt; FIG. 7(B) is a side drawing schematically showing the pulley on which the endless belt is wound; and FIG. 7(C) is a side drawing schematically showing the engagement state of the endless belt with the pulley.

FIG. 8 is a side sectional drawing showing the driving unit.

### DESCRIPTION OF THE PREFERRED DESIGN

Referring to the attached drawings, the details of the design of the present invention will be presented.

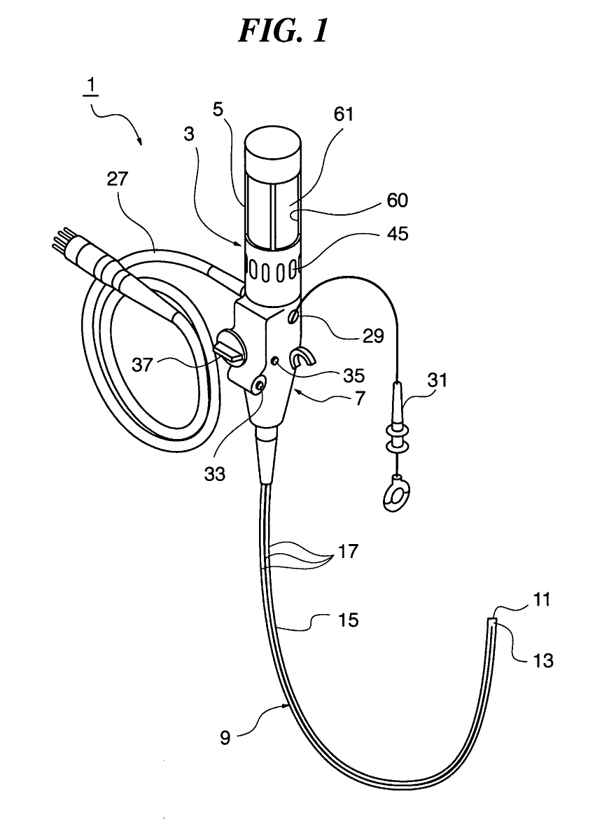

FIG. 1 is a perspective drawing showing the self-propelled colonoscope (example) according to one design of the present invention.

A self-propelled colonoscope 1 is provided with a belt driving unit 5 covered with a driving unit casing 3, an operation unit 7 under the belt driving unit 5, an insertion portion (insertion tube) 9 extending from the operation unit 7 and inserted into the colon, etc. The insertion tube 9 comprises a distal section 11, a bending section 13, and a flexible section (flexible insertion portion) 15. Multiple endless belts 17 are longitudinally arranged on the flexible section 15.

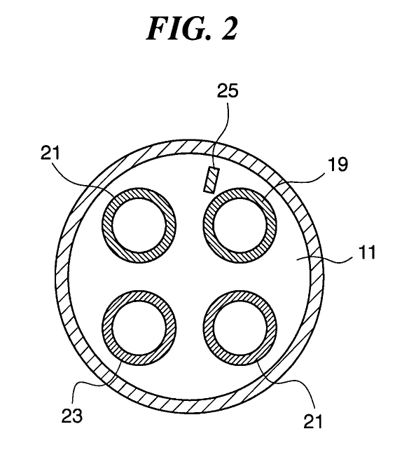

FIG. 2 is a sectional drawing showing the distal section of the insertion portion.

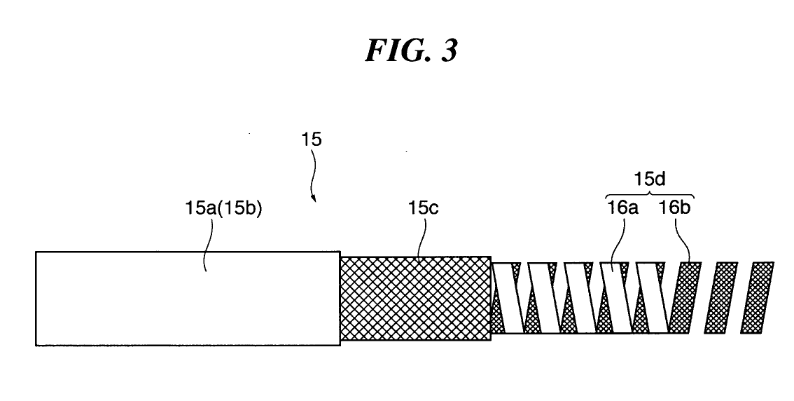

FIG. 3 shows the structure of the flexible tube of the flexible section.

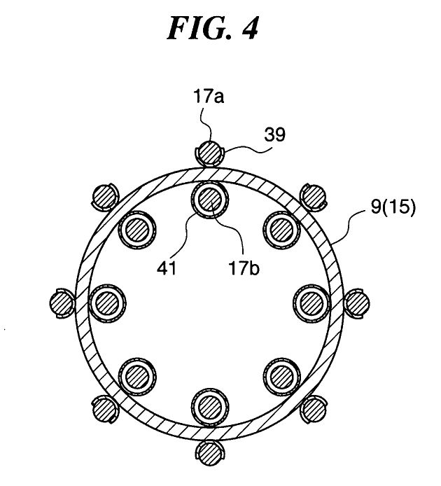

FIG. 4 is a sectional drawing showing the flexible section of the insertion portion.

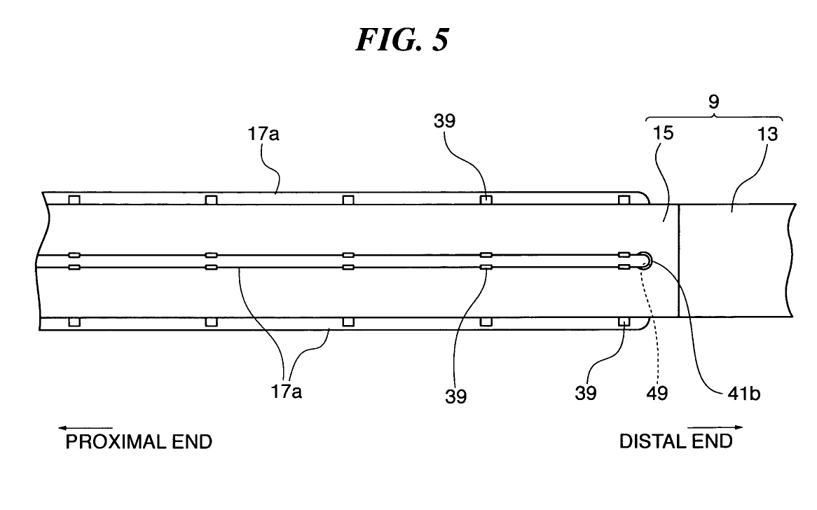

FIG. 5 is a side drawing showing a portion near the distal end of the flexible section.

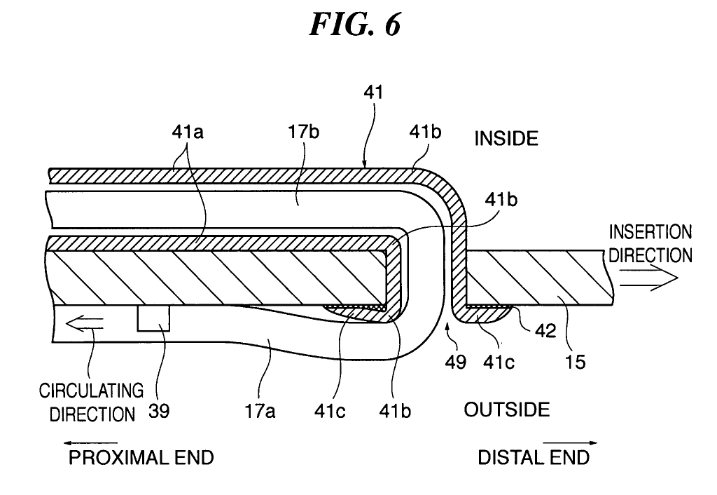

FIG. 6 is an enlarged side sectional drawing showing a portion near the distal end of the flexible section.

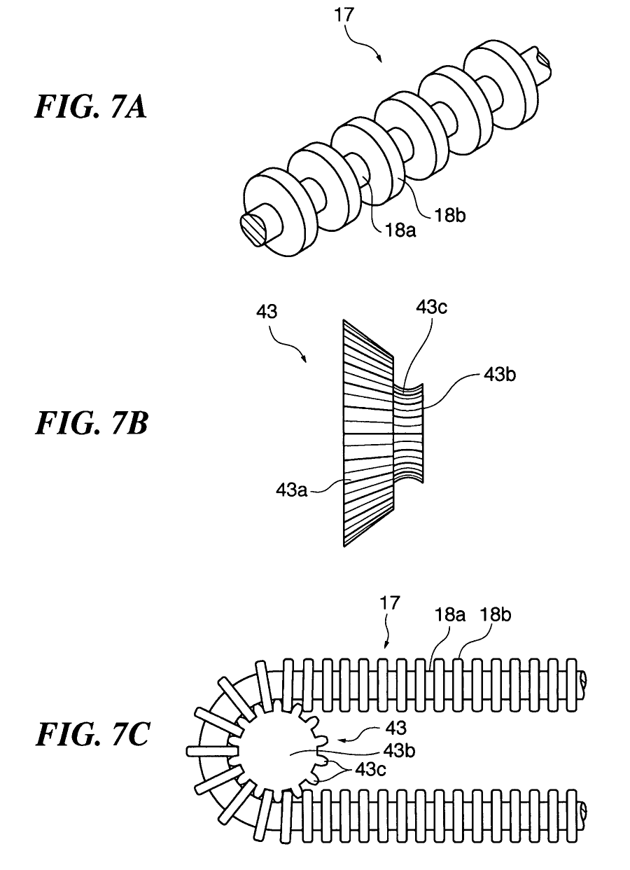

FIG. 7(A) is a perspective drawing schematically showing the structure of the endless belt; FIG. 7(B) is a side drawing schematically showing the pulley on which the endless belt is wound; and FIG. 7(C) is a side drawing schematically showing the engagement state of the endless belt with the pulley.

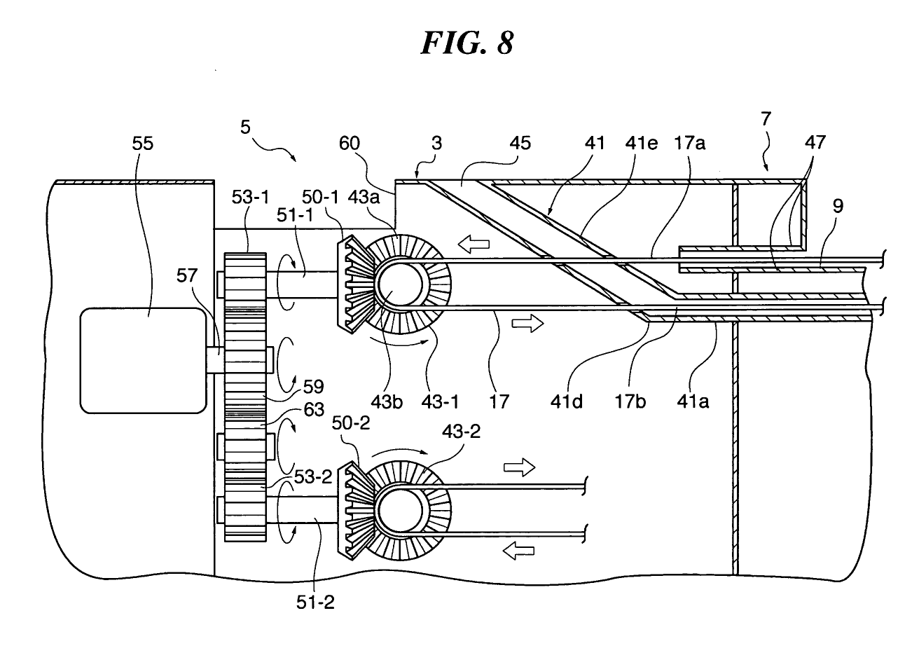

FIG. 8 is a side sectional drawing showing the driving unit.

At the distal section 11 of the insertion tube 9, as shown in FIG. 2, an image receiving window 19, one or two light-projecting windows 21 (two windows in this design), a suction and forceps opening 23, and an air-water nozzle 25 are shown. The image-receiving window 19, equipped with an objective lens when the observation device is a fiberscope, or an image pick-up device, such as a CCD, when it is an electronic scope, receives an image from the distal end surface. The received image is transmitted to the operation unit 7 through the image guide of the fiberscope or the lead wire of the electronic scope, which is inserted into the insertion tube 9, and then transmitted through a universal cord 27 to a display. A light guide, such as an optical fiber, is inserted into the bore of each light-projecting window 21, runs through the operation unit 7 and is connected to an external light source via the universal cord 27. The light source projects light from the distal end surface of the light guide.

The suction and forceps opening 23 is connected to the forceps insertion opening 29 (referring to FIG. 1) in the operation unit 7, and forceps 31 is inserted therein. The working tip end of the forceps 31, protruding from the distal end of the insertion tube 9, is manipulated at the proximal part of the forceps 31 to perform procedures such as to treating a lesion or collecting tissue from a patient.

The bore of the air-water nozzle 25 is a water-air supply channel through which air or a cleaning solution flows and is injected from the air-water nozzle 25 by manipulating the air-water supply button 33 mounted on the operation unit 7. Through the suction and forceps opening 23, bodily fluid or cleaning solution remaining in the colon is sucked out and discharged to outside the patient's body. This operation is carried out by manipulating the suction control button 35 on the operation unit 7.

The flexible section 13 of the insertion tube 9 is able to bend upward and downward, rightward and leftward, and obliquely by manipulating a control knob 37 on the operation unit 7.

The flexible section (flexible insertion portion) of the insertion tube 9 is a flexible tube with a four-layer structure, as shown in FIG. 3, comprising a coating layer 15a, a resin layer 15b, a mesh layer 15c and a flex layer 15d from the outside to the inside in this design. The coating layer 15a serves to provide a smooth feel when inserting of the colonoscope and is made of a heat-resistant, abrasion-resistant and heat-adhesive resin etc. The resin layer 15b serves to protect the mesh layer 15c and the flex layer 15d on the inside and is made of a polyurethane resin. The mesh layer 15c is made by braiding multiple metal or non-metal wires. The flex layer 15d comprises a spirally arranged outer flex 16a and a spirally arranged inner flex 16b. Each flex is made by winding a thin elastic plate, such as a stainless steel plate, into a spiral.

As shown in FIGS. 4, 5, multiple endless belts 17 are longitudinally arranged on the outer wall of the flexible section 15 of the insertion tube 9. The diameter of the flexible section 15 is preferably 5 to 30 mm; within 20 mm is most preferable. A larger number of endless belts 17 is preferable, because the colonoscope has greater self-propelling property as the number of endless belts increases.

The endless belts 17 are arranged on a circulating path around the outer and inner walls of the flexible section 15. On the outer wall of the flexible section 15, each endless belt 17 is supported by guide hooks 39 formed on the outer surface. On the inner wall of the flexible section 15, each endless belt 17 passes through a guide pipe 41 mounted on the inner wall.

The guide hooks 39, each having a circular-arc cross section with a center angle over 180°, are mounted lengthways along the flexible section 15 at suitable intervals, and support the endless belts 17 to be exposed radially and outwardly from the guide hooks 39 (as shown in FIG. 5). Accordingly, the outward surface of the endless belt 17 supported by the guide hooks 39 is exposed from the guide hooks 39 so as to be in contact with the inner wall of the colon with a sufficient contact area when the flexible section 15 is inserted into the colon. Even when the flexible section 15 is severely bent, the endless belts 17 will not come off the guide hooks 39.

The guide hooks 39 are mounted lengthways along the flexible section 15 at intervals of 2 to 3 cm, in this design; however, the guide hooks 39 may be continuously placed in the lengthways direction of the flexible section. The guide hook 39 is made of an elastic material to spread the opening thereof.

The guide pipe 41 is made of a flexible thin-walled metal tube, a tube made of ethylene tetrafluoride or a closely winded coil pipe. As shown in FIG. 6, the guide pipe 41 has a straight section 41a extending straight in the flexible section 15; a bent section 41b bent outside at an angle of 90° or more from the distal end of the straight section 41a; and a flange section 41c formed at the distal end. The straight section 41a is arranged lengthways along the inner wall of the flexible section 15. The bent section 41b bends outwardly from the distal end of the straight section 41a, passes through a guide hole 49 penetrating the flexible section 15 from inside to outside at the distal end of the flexible section 15, and appears on the outer wall of the flexible section 15. The flange section 41c adheres to the outer wall of the flexible section 15 on the inner surface (under surface) with an adhesive 42. In order to reduce the friction of the endless belts 17 with the flange section 41c, which extends outwardly from the distal end of the bent section 41b, the angle between the bent section 41b and the flange section 41c is set to 90° or larger. Owing to the width of the flange section 41c, the contact area of the flange section 41c with the outer wall of the flexible section 15 becomes larger, improving the seal. The material of the adhesive 42 is selected according to the material of the flex tube of the flexible section 15. By appropriate selection of resin materials for the guide pipe 41 and the resin layer 15b (shown in FIG. 3) of the flexible section 15, the guide pipe 41 and the resin layer 15b are adhered by a heat seal.

As shown in FIGS. 4 and 5, when the colonoscope is inserted, the endless belts 17, supported by the guide hooks 39 mounted on the outer wall of the flexible section 15, run in the opposite direction to the insertion direction of the colonoscope while remaining in contact with the inner wall of the colon on the outside of the flexible section 15. This propels the colonoscope into the colon and, on the inside of the flexible section 15, the endless belts 17 run in the insertion direction through the straight section 41a of the guide pipe 41 extending in the lengthways direction of the flexible section 15. As shown in FIG. 6, at the distal end of the flexible section 15, the endless belts 17 exits the straight section 41a through the bent section 41b and the flange section 41c adhered to the guide hole 49.

When the colonoscope is removed from the patient's body, the endless belts 17 run in the opposite direction to the direction at insertion; that is, the endless belts 17 running in the insertion direction of the colonoscope on the outside of the flexible section 15, run in the opposite direction to the insertion direction on the inside of the flexible section 15.

As described above with reference to FIG. 3, the flexible section 15 has a four-layer structure comprising the coating layer 15a, the resin layer 15b, the mesh layer 15c and the outer and inner flex layer 15d in this design. Since the mesh layer 15c and the outer and inner flex layer 15d are not solid but porous, liquid may easily infiltrate the layers from the sidewall of the guide hole 49 penetrating the flexible section 15. However, in this design, the guide pipe 41 passes through the guide hole 49 at the distal end of the flexible section 15, and the flange section 41c formed at the distal end of the guide pipe 41 appears on the outside and adheres to the outer wall of the flexible section 15. This prevents the mesh layer 15c and the flex layer 15d from being exposed to the sidewall of the guide hole 49. Accordingly, when the endless belts are driven, the layers are not infiltrated with bodily fluid adhered to the endless belts from the sidewall of the guide hole 49. Similarly, when the body of the colonoscope is cleaned after removing the endless belts, the layers are also not infiltrated with cleaning liquid from the sidewall.

Each of the endless belts 17 is made of a flexible and strong material such as, for instance, carbon fiber or resin and, as shown in FIG. 7(A) and FIG. 7(C), each comprises an axial belt 18a and multiple rack gear teeth 18b arranged lengthways along the axial belt 18a, which has a circular cross section with a diameter of 1 to 3 mm. The rack gear teeth 18b also have a circular cross section and are coaxially arranged at regular intervals on the outer surface of the axial belt 18a. The rack gear teeth 18b have a diameter of 1 to 3 mm and a thickness of 0.1 to 1.0 mm and the distance between any two of the rack gear teeth 18b is 0.1 to 1.0 mm. The diameters of the axial belt 18a and the rack gear teeth 18b are selected within these ranges so that the diameter of the rack gear teeth 18b is larger than the diameter of the axial belt 18a. The outer surface of the rack gear teeth 18b may be coated with high frictional material, and the outer surface of the pulley 43c including pinion gear teeth 43c, as described later, may be also coated with high frictional material.

The length of the endless belt 17 will be described later, but it is formed by adhering the ends of a single belt. When the colonoscope is cleaned, the endless belt 17 is detached at the intermediate portion and removed from the body of the colonoscope. The method for removing and mounting the endless belt is not described.

The endless belt has a circular cross section so as to be able to bend flexibly in all radial directions to the axis with equal force. So, when the insertion tube 9 is inserted around the bends of the colon, the endless belt 17 can easily follow the motion of the insertion tube 9. Since the rack gear teeth 18b are formed along the entire length of the endless belt 17, if the endless belt 17 is twisted, the rack gear teeth 18b may be in contact with the inner wall of the colon, causing the endless belt 17 to rub against it. Accordingly, the friction force between the endless belts 17 and the inner wall of the colon will be increased to improve the self-propelling ability.

Next, referring to FIG. 8, the construction of the proximal part of the guide pipe 41 and the belt driving unit 5 will be explained.

The proximal part of the guide pipe 41 is connected to a guide-pipe opening 45 in the side surface of the driving unit casing 3, which has a larger diameter than the insertion tube 9. The straight section 41a of guide pipe 41, extending straight in the flexible section 15 from the distal to the proximal end, is connected to an inclined section 41e, extending obliquely and outwardly, via a bent section 41d in the driving unit casing 3 and then to the guide-pipe opening 45.

The drive roller 43 to which the endless belt 17 is attached is found at a proximal side of the guide pipe 41 in the driving unit casing 3. The exterior portion 17a of the endless belt 17 passes through a guide section 47 from the outside of the insertion tube 9 and enters the driving casing 3 through holes penetrating across the inclined section 41e of the guide pipe 41. The endless belt 17 is wound around the drive roller 43. Then, the interior portion 17b of the endless belt 17 enters the straight section 41a of the guide pipe 41 through a hole in the inclined section 41e and then extends through the straight section 41a toward the guide hole 49 (as shown in FIGS. 5 and 6) formed at the distal end of the flexible section 15 of the insertion tube 9.

The guide hole 49, as shown in FIG. 5, is preferably positioned 0 to 10 cm from the distal end of the flexible section 15. This is because the larger the area where the inner wall of the colon is in contact with the exterior portion 17a of the endless belts 17, the better the self-propelling ability of the colonoscope becomes.

The insertion tube 9, inserted into the colon, advances from the sigmoid colon to the ileum through the descending colon, the transverse colon and the ascending colon in the above described manner. Since the flexible section 15 has an outer diameter as great as about 16 mm, when the colonoscope advances into the colon, the curve of the colon causes a difference in the inner ring length and the outer ring length of the inserted flexible section 15. For example, when the distal end of the insertion tube 9 reaches the ileum and the flexible section 15, having a diameter of 16 mm, turns in a curve, the outer ring length is 3.12% longer than the straight length.

Accordingly, the endless belt 17 on the outer and inner walls of the flexible section 15 necessarily has an allowable length for differences in the inner and outer ring length of the flexible section 15. For this reason, the length of the endless belt 17 is designed be 102 to 104% of the length of the belt when it turns under tension from the guide hole 49 formed at the distal end of the flexible section 15 to the same guide hole 49 through the driving unit while keeping the flexible section 15 straight. Since the length of the endless belt 17 is set as above, it can sufficiently follow the bending of the flexible section 15; therefore, the colonoscope can be propelled into the colon stably.

Referring again to FIG. 8, the drive roller 43 is provided with a pulley 43b on which the endless belt 17 is wound and a bevel gear 43a is connected to the same shaft as the pulley 43b. On the circumferential surface of the pulley 43b, as shown in FIG. 7(C), a concave groove is formed, in which the pinion gear teeth 43c are fixed, which engage with the rack gear teeth 18b of the endless belt 17.

As mentioned above, the length of the endless belt 17 has a certain allowance; however, since the pulley 43b for driving the endless belt 17 is formed with the pinion gear teeth 43c on the circumferential surface, the endless belt 17 and the pulley 43b are tightly engaged with each other by engagement of the rack gear teeth 18b and the pinion gear teeth 43c, whereby the endless belt 17 can be driven without idling.

As shown in FIG. 8, a bevel gear 50 is arranged orthogonally to the bevel gear 43a and engaged therewith. A spur gear 53 is fixed at the proximal end of the gear shaft 51 of the bevel gear 50. The spur gear 53 is engaged with a large spur gear 59 fixed to the motor shaft 57 of the motor 55. Consequently, when the motor shaft 57 revolves by driving the motor 55, the bevel gear 43a will revolve, via the large spur gear 59, the spur gear 53, and the bevel gear 50, and therefore the pulley 43b will also revolve.

On the circumferential surface of the large spur gear 59, the same number of drive rollers 43, bevel gears 50, gear shafts 51, and spur gears 53, as the endless belts 17, are mounted. Incidentally, an intermediate gear 63 may be provided between the large spur gear 59 and the spur gear 53 in order to drive each of the endless belts 17 in the same direction.

The motor 55, the large spur gear 59, the spur gear 53, the gear shaft 51, the bevel gear 50, and the drive roller 43 are housed in the driving unit casing 3 on the proximal side of the guide pipe opening 45. On the side surface of the driving unit casing 3, a cleaning opening 60 is formed, which is provided with a lid 61 for opening and closing the opening 60, as shown in FIG. 1. The opening 60 opens to a space containing the large spur gear 59, the spur gear 53, the gear shaft 51, the bevel gear 50, and the drive roller 43. The space containing the large spur gear 59, the spur gear 53, the gear shaft 51, the bevel gear 50, and the drive roller 43 is liquid-tight and isolated from the space containing the motor 55.

When the motor 55 is driven to rotate the pulley 43b counterclockwise, the exterior portion 17a of the endless belt 17 engaged with the pulley 43b will run to the left direction in the figure. When the exterior portion 17a of the endless belt 17 is in contact with the inner wall of the colon, the insertion tube 9 will be propelled forward to the right direction in FIG. 8 by friction between the endless belts 17 and the inner wall of the colon. The insertion tube 9 will be propelled back by rotating the motor 55 clockwise.

1. A self-propelled colonoscope comprising:
a tubular flexible insertion portion inserted into the colon;
an endless belt arranged on a circulating path around the outer and inner walls of the flexible insertion portion;
a driving mechanism to drive the endless belt; and
a guide pipe extending along the circulating path and guiding the endless inside the flexible insertion portion, wherein the end of the guide pipe penetrates through the tube wall of the flexible insertion portion to the outside at the distal end of the flexible insertion portion.

2. In the self-propelled colonoscope according to claim 1,
the end of the guide pipe is formed into a flange shape, the end of which adheres to the outer wall of the flexible insertion portion.

3. A self-propelled endoscope comprising:
a tubular flexible insertion portion inserted into an object;
an endless belt arranged on a circulating path around the outer and inner walls of the flexible insertion portion;
a driving mechanism to drive the endless belt; and
a guide pipe extending along the circulating path and guiding the endless belt inside the said flexible insertion portion, wherein the end of the guide pipe penetrates through the tube wall of the flexible insertion portion to the outside at the distal end of the flexible insertion portion.
